(19) United States
(12) Patent Application Publication
(10) Pub. No.: US 2014/0039237 A1
(43) Pub. Date: Feb. 6, 2014

(54) MEDICATION DISPENSING DEVICE

(71) Applicant: MING-HSIN LI, Taoyuan County (TW)

(72) Inventor: MING-HSIN LI, Taoyuan County (TW)

(21) Appl. No.: 13/567,107

(22) Filed: Aug. 6, 2012

(51) Int. Cl. A61M 36/06

(52) U.S. Cl. 600/4

(57) ABSTRACT

A medication dispensing device includes a medication bottle, a perfusion device, a filter and a dispensing needle. The dispensing needle includes a lower end being shrill, smooth and seamless and puncturing into the plug of the medication bottle. The dispensing needle includes a passageway therein, and includes an opening and a groove on the side wall of the dispensing needle. The dispensing needle can puncture into the plug of the medication bottle without creating any scraps stuck in the opening of the passageway or dropping into the medication bottle. The groove can release the air and pressure of the medication bottle when the pressure in the medication bottle increases during the injection of the medication solution. Accordingly, the smoothness of the dispensation of the medication solution is improved, and the contamination of the medication solution can be avoided.

### BACKGROUND OF THE INVENTION

1. Field of the Invention

The present invention is related to a medication dispensing device, and more particularly to a filling dispensing needle applied in dispensation of nuclear medicine that can keep the medication from being polluted.

2. Description of the Prior Arts

For imaging techniques of cancer diagnosis, the injection liquid of radioactive drugs used in positron emission tomography (PET) or single photon emission tomography (SPECT) is required to be dispensed. Such technique is generally applied in the research and development of radiopharmaceuticals in nuclear medicine and dispensation process thereof, as well as the research and development of drugs in biotechnology.

With reference to FIG. 6, a conventional medication dispensing device comprises a medication bottle 60, a perfusion device 61, a filter 62, a dispensing needle 63 and an aeration needle 64. The prepared medication solution is stored in the medication bottle 60. The medication bottle 60 has an opening and a plug 600 in the opening. The perfusion device 61 is a pump, and has an inlet and an outlet. The inlet is connected to an import pipe 610. The import pipe 610 can be arranged in the medication solution (such as radioactive solution). The outlet is connected to an export pipe 611. The filter 62 is connected to an injection tube 620 and the export pipe and has a film mounted therein. The dispensing needle 63 is a cylinder hollow tube and has two openings at two ends respectively. One of the two ends of the dispensing needle 63 is a tip end. The tip end of the dispensing needle 63 punctures the plug 600 of the medication bottle 60, and the opening of the other end of the dispensing needle 63 is connected to the injection tube 620. Similarly, the aeration needle 64 is also a cylinder hollow tube and has two openings at two ends respectively. One of the two ends of the aeration needle 64 is also a tip end. The tip end of the aeration needle 64 punctures the plug 600 of the medication bottle 60.

Such medication dispensing device is required to operate in an environment in compliance with Class 100 laminar air ventilation. The import pipe 610 of the perfusion device 61 is attached in a prepared radioactive solution. The aeration needle 64 can balance the pressure between the inside and the outside the medication bottle 60 and, such that the medication bottle 60 will not burst along addition of the amount of the solution. However, as the dispensing needle 63 and the aeration needle 64 puncture into the plug 600 by the tip ends thereof, some scraps of the plug are created and stuck in the openings of the tip ends. Moreover, in the dispensing process, the scraps drop down to the medication bottle 60 that causes contamination therein. The configuration of the aeration needles 64 is easily resulting in direct connection between the inside and the outside of the medication bottle 60, such that the inside of the medication bottle 60 are more easily contaminated by external impurities and more difficult to keep aseptic. Therefore, in order to avoid the contamination in the medication bottle 60, the operation environment is need to be strictly controlled that makes the demands and costs of the equipment and condition mush higher, and the quality is hard to maintain. Also, the dispensing needle 63 can have a channel with a triangle section on the side wall thereof, and the channel is used for balancing pressure, and yet the problem about the scraps still exists.

To overcome the shortcomings, the present invention provides an improved medication dispensing device to mitigate and obviate the aforementioned problems.

### SUMMARY OF THE INVENTION

In view of foregoing problems, since the scraps can be created and dropped down to the medication bottle and the inside and the outside of the medication bottle are connected directly when the dispensing needle and the aeration needle punctures into the plug, which cause the medication bottle is hard to keep aseptic inside due to contamination, the purpose of present invention is to provide a medication dispensing device to avoid the external contamination toward the medication bottle by improvement of the structure of the dispensing needle.

In order to achieve the objective, a medication dispensing device in accordance with the present invention is provided, and comprising:
a medication bottle provided to store prepared medication solution, and having an opening and a plug installed in the opening;
a perfusion device having an import pipe and an export pipe;
a filter having a film and an injection tube, wherein the filter is connected to the export pipe; and
a dispensing needle being a hollow tube and having:
   an upper end connected to the injection tube;
   a lower end being shrill, smooth and seamless and puncturing into the plug of the medication bottle; and
   passageway formed therein and having a first opening and a second opening, wherein the first opening formed through the top surface of the dispensing needle, and the second opening formed through the side wall of the dispensing needle.

Preferably, the plug is elastic, and the dispensing needle has a groove along the axial direction on the side wall of the dispensing needle and wrapped around tightly by the plug to form a pressurized seal part.

Preferably, the film of the filter has multiple pores with 0.22 μm of pore diameter to filter out particles and bacteria.

Preferably, the medication bottle, the perfusion device, the filter and the dispensing needle are arranged in an environment in compliance with Class 100 laminar air ventilation.

Preferably, the import pipe and the export pipe are made of material proof of acid and alkali.

A dispensing needle in accordance with the present invention is also provided, being a hollow tube and comprising:
an upper end;
a lower end being shrill, smooth and seamless; and
a passageway formed therein and having a first opening and a second opening, wherein the first opening formed through the top surface of the dispensing needle, and the second opening formed through the side wall of the dispensing needle.

Preferably, the second opening is formed close to the side wall of the lower end, thereby the second opening is located beneath the plug when the lower end punctures into the plug.

Preferably, the dispensing needle has a groove along the axial direction on the side wall of the dispensing needle.

Preferably, the shape of the section of the groove is triangle.

Accordingly, due to the injection of the medication solution into the medication bottle through the second opening of the dispensing needle, the process of puncturing aeration needle is not required, and the contamination of the medication solution by the external pollutant through the aeration needle or the opening of the medication bottle are reduced. The lower end of the dispensing needle is shrill, smooth and seamless, such that the dispensing needle can puncture into the plug of the medication bottle without creating any scraps, and thus the problem about scraps dropping into the medication bottle can be overcome. When the medication solution is injected into the medication bottle along the passageway of the dispensing needle, the vacuity of the medication bottle is decreasing to achieve the balance of pressure. When the medication solution stops injected, the plug will wrap the second opening of the dispensing needle, and air (including bacteria, particles, and so on) cannot flow freely along the passageway. Thus, the medication solution can be assured to be kept from external contamination, the demands of the condition and equipment can be made less, the quality can be controlled easily, and the costs can be reduced.

Since the groove is formed on the side wall of the dispensing needle, when the dispensing needle punctures into the plug in the operation of the medication dispensing device, the plug with elasticity can wrap tightly around the dispensing needle and the groove and form in a pressurized seal part that has less pressure. The pressurized seal part has a general effect of obstruction. When the pressure in the medication bottle increases to a default value with the addition of radioactive drug, the pressurized seal part with weaker strength will be forced to deform by the pressure, and thus the inside and the outside of the medication bottle are interconnected through the groove for releasing the accumulated air and pressure inside the medication bottle, thereby the smoothness of the dispensing process can be improved and the drugs can be kept from external contamination.

Other objectives, advantages and novel features of the invention will become more apparent from the following detailed description when taken in conjunction with the accompanying drawings.

### BRIEF DESCRIPTION OF THE DRAWINGS

FIG. 1 is a front view of a first embodiment of a medication dispensing device in accordance with the present invention.

FIG. 2 is a side view in partial section of a first embodiment of a medication dispensing device in accordance with the present invention.

FIG. 3 is a top view in partial section of a first embodiment of a medication dispensing device in accordance with the present invention.

FIG. 4 is a side view in partial section of a second embodiment of a medication dispensing device in accordance with the present invention.

FIG. 5 is a top view in partial section of a second embodiment of a medication dispensing device in accordance with the present invention.

FIG. 6 is a front view of a medication dispensing device in accordance with the prior art.

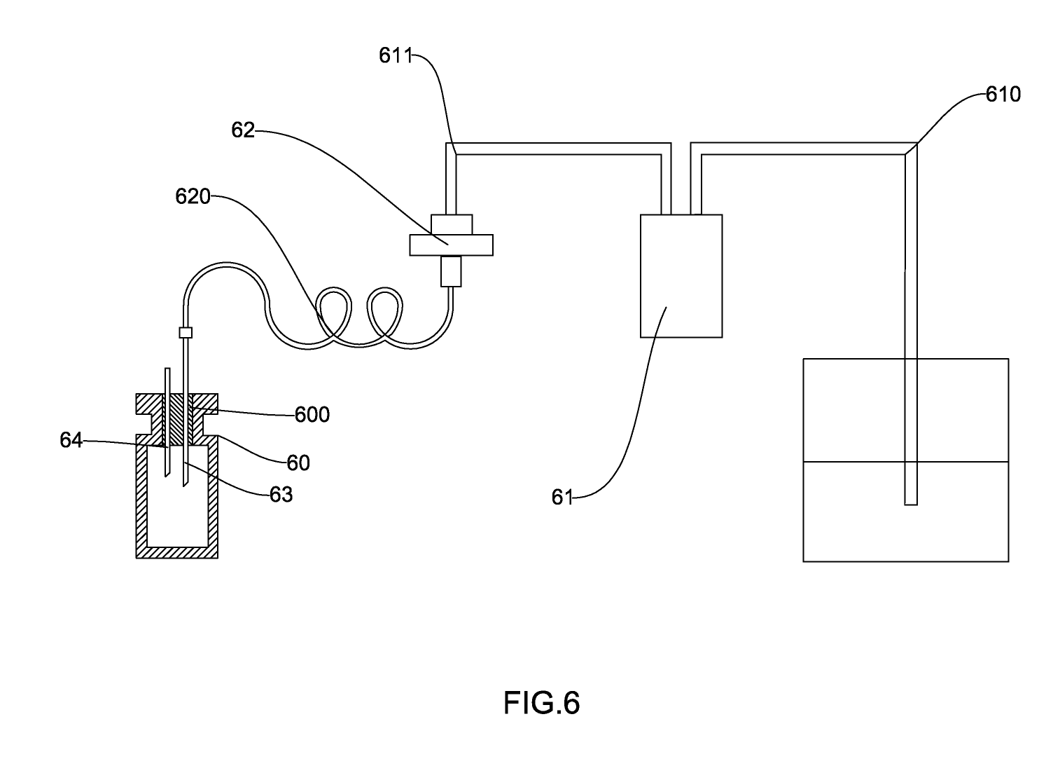

### DETAILED DESCRIPTION OF THE INVENTION

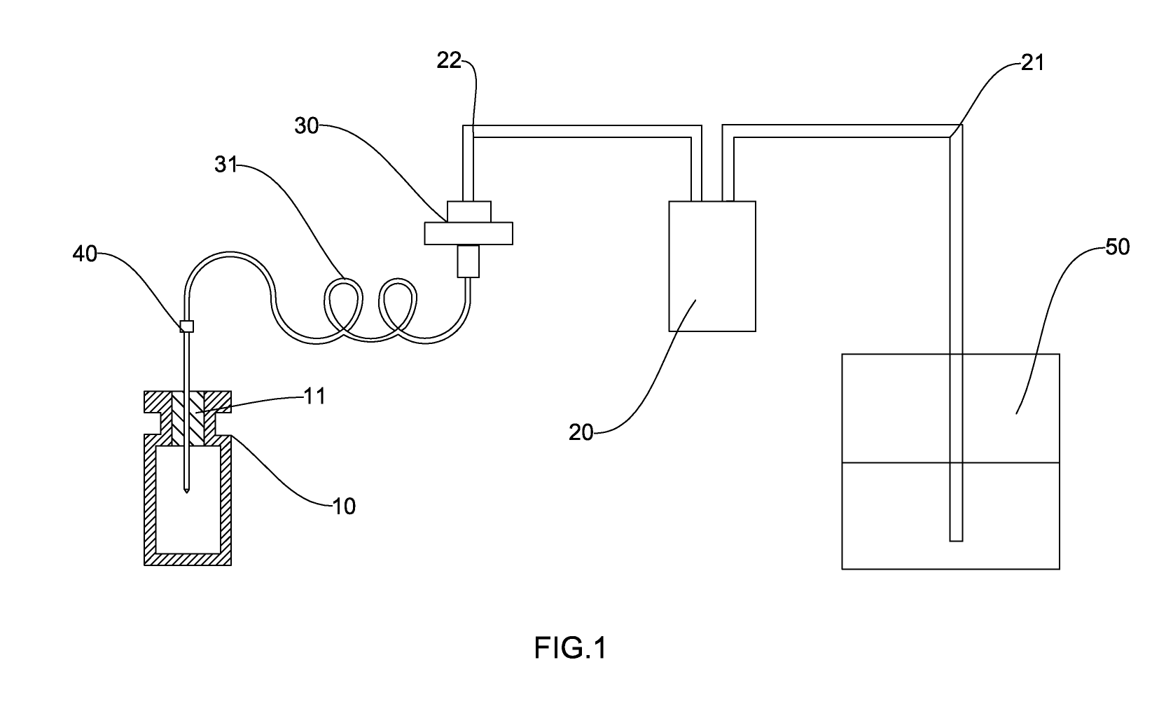

With reference to FIG. 1, the medication dispensing device comprises a medication bottle 10, a perfusion device 20, a filter 30 and a dispensing needle 40.

The medication bottle 10 is a container, the medication bottle 10 provides to store prepared medication solution. The medication bottle 10 has an opening and a plug 11 installed in the opening. The plug 11 is elastic including corkwood or rubber.

The perfusion device 20 is a pump which has an import and an export. The import is connected to an import pipe 21, and the export is connected to an export pipe 22. The import pipe 21 and the export pipe 22 are made of material proof of acid and alkali. The import pipe 21 and the export pipe 22 are made of glass in the preferred embodiment.

The filter 30 has a film and an injection tube 31. The film has multiple pores with 0.22 μm of pore diameter. The filter 30 is connected to the export pipe 22.

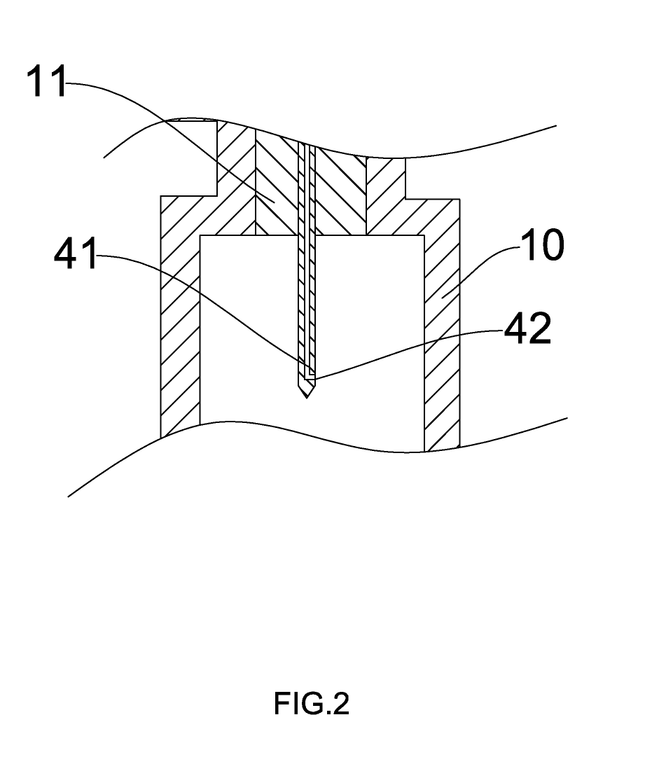

With reference to FIG. 2, the dispensing needle 40 is a hollow tube made of stainless steel. The dispensing needle 40 has an upper end, a lower end and a passageway 41. The lower end is shrill, smooth and seamless. The passageway 41 is formed in the dispensing needle 40 and has a first opening and a second opening 42. The first opening is formed through the top surface of the dispensing needle 40. The second opening 42 is formed through the side wall of the dispensing needle 40 and formed close to the side wall of the lower end.

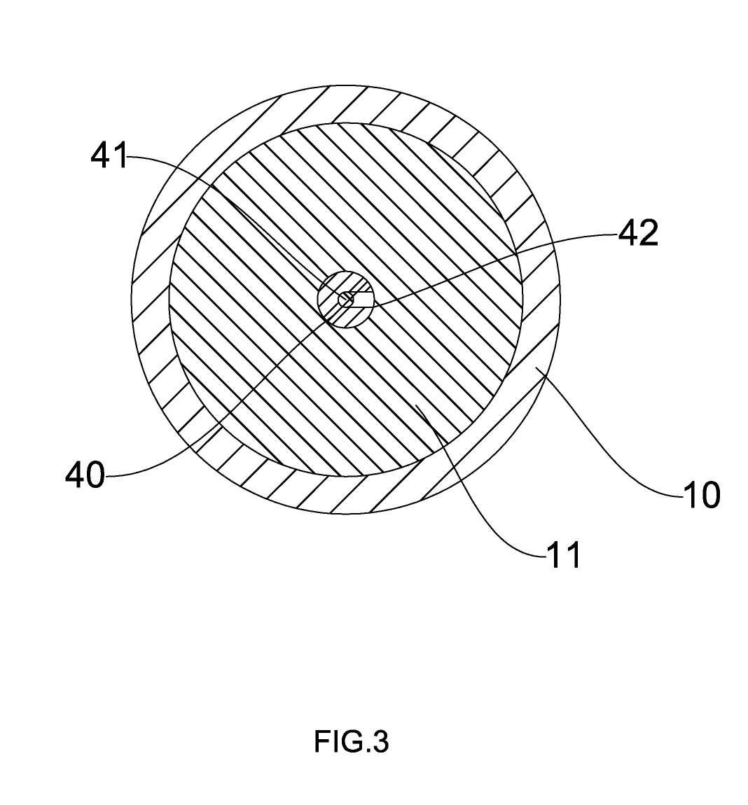

The upper end is connected to the injection tube 31 of the filter 30. The lower end punctures into the plug 11 of the medication bottle 10. Since the lower end is shrill, smooth and seamless, the dispensing needle 40 can puncture into the plug 11 without creating any scraps. With reference to FIG. 3 for a section view of the plug 11 and the dispensing needle 40, the plug 11 that has elasticity wraps tightly around the side wall of the dispensing needle 40 and forms in an airproof part.

During the dispensing process, the medication dispensing device is arranged in an environment in compliance with Class 100 laminar air ventilation. The medication solution, such as radiopharmaceutical solution, is prepared in an apyrexia receptacle.

The import pipe 21 of the perfusion device 20 is attached into the medication solution of the apyrexia receptacle. The medication solution is pumped by the perfusion device 20 and passing through the import pipe 21, and then delivered to the filter through the export pipe 22 under pressure. After passing through the filter 30 to be filtered out particles and bacterium, the medication solution is delivered into the passageway 41 of the dispensing needle 40 via the injection tube 31 and then delivered to the medication bottle 10 through the second opening 42 of the passageway 42 to form a radioactive injection.

Accordingly, the medication solution can be injected into the medication bottle 10 through the second opening 42 of the dispensing needle 40. The lower end of the dispensing needle 40 is shrill, smooth and seamless, and thus the creation of scraps can be avoid during the puncture of the dispensing needle. Also, the problem of the scraps drooping into the medication bottle 10 can be solved. Furthermore, when the medication bottle 10 is filled with the medication solution by the dispensing needle 40, the vacuity in the medication bottle 10 gradually decreases during dispensing the medication solution and reach the balance of pressure. when stopping dispensing the medication solution, the plug 11 can wraps around the second opening 42, such that air (bacteria, particles, and so on) cannot flow freely through the passageway 11.

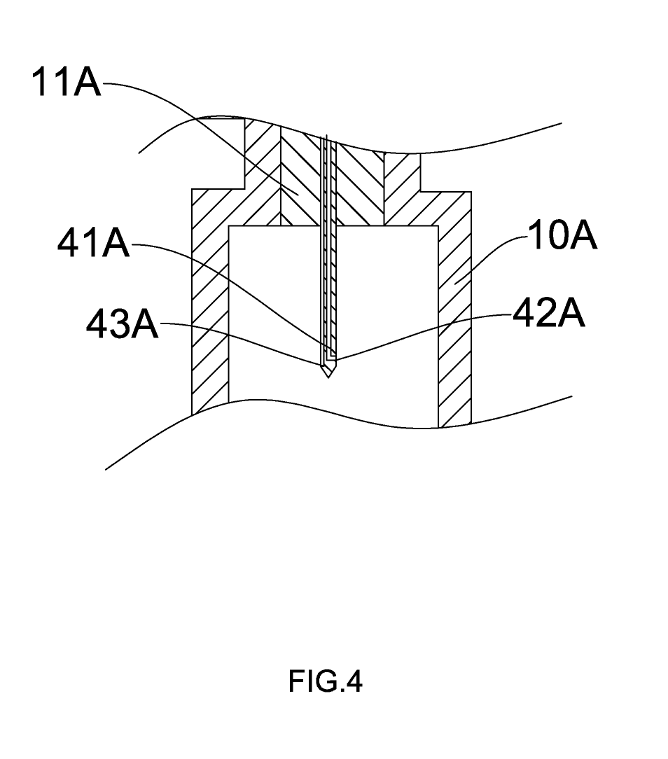

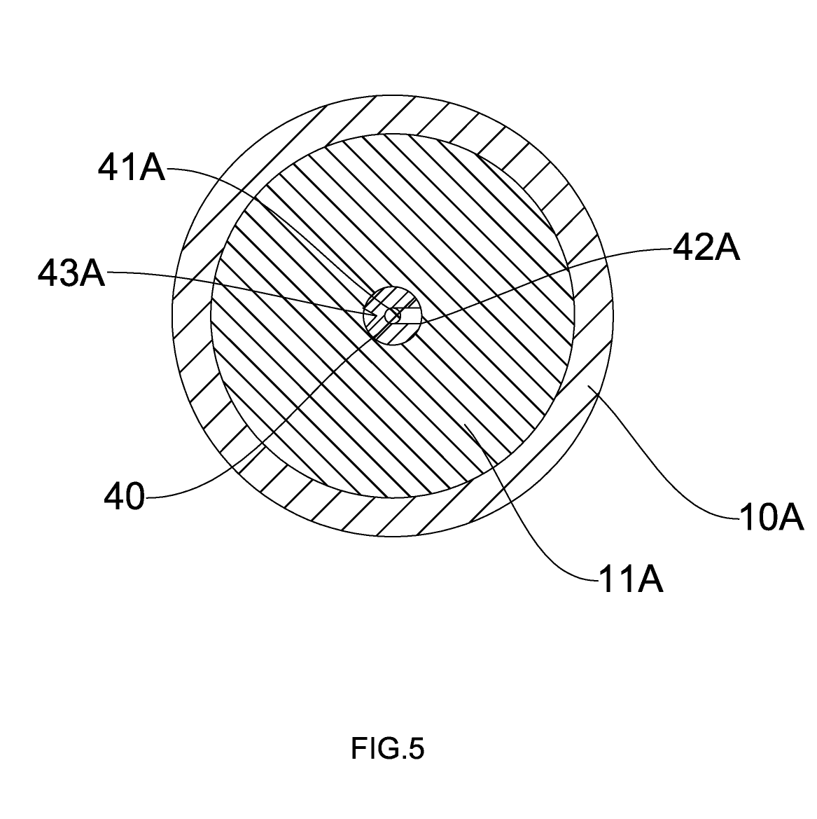

With reference to FIGS. 4 and 5, the medication bottle 10A is not in a vacuum state. In one aspect, a groove 43A along the axial direction of the dispensing needle 40A is formed on the other side wall of the dispensing needle 40A and having a section in a shape of triangle.

During the operation of the medication dispensing device, when the dispensing needle 40A punctures into the plug 11A, the plug 11A that has elasticity can wrap tightly around the dispensing needle 40A and the groove 43A and form in a pressurized seal part that has less pressure. The pressurized seal part has a general effect of obstruction. When the pressure in the medication bottle 10A increases to a default value with the addition of radiopharmaceutical drugs, the pressurized seal part with weaker strength will be forced to deform by the pressure, and thus the inside and the outside of the medication bottle 10A are interconnected through the groove 43A for releasing the accumulated air and pressure inside the medication bottle 10A, thereby the smoothness of the dispensing process can be improved and the radiopharmaceutical drugs can be kept from external contamination.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A medication dispensing device comprising:
a medication bottle provided to saved prepared medication solution and having an opening and a plug installed in the opening;
a perfusion device having an import pipe and an export pipe;
a filter having a film and an injection tube, wherein the filter is connected to the export pipe; and
a dispensing needle being a hollow tube and having:
an upper end connected to the injection tube;
a lower end being shrill, smooth and seamless and puncturing into the plug of the medication bottle; and
a passageway formed therein and having a first opening and a second opening, wherein the first opening formed through a top of the dispensing needle, and the second opening formed through the side wall of the dispensing needle.

2. A medication dispensing device as claimed in claim 1, wherein the plug is elastic, and the dispensing needle has a groove along the axial direction on the side wall of the dispensing needle and wrapped around tightly by the plug to form a pressurized seal part.

3. A medication dispensing device as claimed in claim 1, wherein the film of the filter has multiple pores with 0.22 μm of pore diameter to filter out particles and bacteria.

4. A medication dispensing device as claimed in claim 2, wherein the film of the filter has multiple pores with 0.22 μm of pore diameter to filter out particles and bacteria.

5. A medication dispensing device as claimed in claim 1, wherein the medication bottle, the perfusion device, the filter and the dispensing needle are arranged in an environment in compliance with Class 100 laminar air ventilation.

6. A medication dispensing device as claimed in claim 5, wherein the import pipe and the export pipe are made of material proof of acid and alkali.

7. A dispensing needle, being a hollow tube and comprising:
an upper end connected to the injection tube;
a lower end being shrill, smooth and seamless and puncturing into the plug of the medication bottle; and
a passageway formed therein and having a first opening and a second opening, wherein the first opening formed through a top of the dispensing needle, and the second opening formed through the side wall of the dispensing needle.

8. A dispensing needle as claimed in claim 7, wherein the dispensing needle has a groove along the axial direction on the side wall of the dispensing needle.

9. A dispensing needle as claimed in claim 8, wherein the shape of the section of the groove is triangle.

10. A dispensing needle as claimed in claim 8, wherein the second opening is formed close to the side wall of the lower end, thereby the second opening is located beneath the plug when the lower end punctures into the plug.
